(19) United States
(12) Patent Application Publication
(10) Pub. No.: US 2001/0007659 A1
(43) Pub. Date: Jul. 12, 2001

(54) USE OF LENTIVIRAL VECTORS FOR ANTIGEN PRESENTATION IN DENDRITIC CELLS

(75) Inventors: FLOSSIE WONG-STAAL, SAN DIEGO, CA (US); XINGIANG LI, SAN DIEGO, CA (US); JUNE KAN-MITCHELL, RANCHO SANTA FE, CA (US)

Correspondence Address: LISA A. HAILE, PH.D., GARY CARY WARE & FREIDENRICH, LLP, 4365 EXECUTIVE DRIVE, SUITE 1600, SAN DIEGO, CA, 92121-2189, US

(73) Assignee: THE UNIVERSITY OF CALIFORNIA

(21) Appl. No.: 09/061,986

(22) Filed: Apr. 17, 1998

Related U.S. Application Data
(60) Provisional application No. 60/043,264, filed on Apr. 17, 1997.

(51) Int. Cl. A61K 48/00; C12N 15/00; C12N 15/63

(52) U.S. Cl. 424/93.21; 435/320.1; 424/93.2; 514/44

(57) ABSTRACT

The present invention provides methods for inducing immunity in a subject by using dendritic cells transduced with a lentivirus vector constructed to deliver an antigenic epitope. The methods of the invention are particularly suited to inducing immunity to human immunodeficiency virus (HIV) and other viral diseases, as well as to inducing immunity to tumor antigens.

### CROSS REFERENCE TO RELATED APPLICATIONS

This application claims priority from U.S. Provisional Application Serial No. 60/043,264 filed Apr. 17, 1997.

### STATEMENT AS TO FEDERALLY SPONSORED RESEARCH

This invention was made in part with Government support under Grant No. AI36612 awarded by the National Institutes of Health. The Government has certain rights in this invention.

### FIELD OF THE INVENTION

The present invention relates generally to the field of immunology and induction of immune responses and more specifically to the use of dendritic cells transduced with a lentivirus vector constructed to deliver an antigenic epitope for inducing immunity.

### BACKGROUND OF THE INVENTION

The host immune system provides a sophisticated defense mechanism which enables the recognition and elimination of foreign entities, such as infections agents or neoplasms, from the body. When functioning properly, an effective immune system distinguishes between foreign invaders and the host's own tissues. The ability to specifically ignore the host's own tissues is called immune tolerance. Immune tolerance to self normally develops at birth when self antigens are brought to the thymus by antigen presenting cells (APCs). APCs play a crucial role in the “programming” of the immune system by specifically indicating which antigens are considered foreign, and thereby, are targeted by the immune system.

Dendritic cells (DCs) are efficient antigen presenting cells (APC) that initiate immune response to peptide antigens associated with class I and II MHC (Freudenthal, P. S. and Steinman, R. M., Proc. Natl. Acad. Sci. USA 87:7698, 1990; Steinman, R. M., Ann. Rev. Immunol. 9:271, 1991). DCs represent a small subpopulation of widely distributed, bone-marrow-derived leucocytes, which are the only natural antigen presenting cells able to prime naive T cells. They activate both CD4+ and CD8+ T lymphocyte primary immune response, and are at least as effective as other APCs such as monocytes in stimulating secondary immune responses (Peters et al., Immunol. Today 17:273, 1997). In lymphoid tissues, the DC are primarily localized in the T cell areas. The B cell areas or follicles of lymphoid organs contain a second type of DC, the Follicular Dendritic Cell (FDC).

Several populations of human DC have been identified from the peripheral blood. These include the myeloid DC which can be produced from precursors after in vitro culture with GM-CSF and IL-4. The latter cytokine appeared to be necessary to inhibit emergence of monocytes/macrophages. Functionally and phenotypically, mature DC were identified among other cell types after expansion of proliferative CD34+ progenitors in GM-CSF and TNFα. Large numbers of fully functional DC have been generated from purified, adherent monocytes (mo-DC) cultured in GM-CSF and IL-4 (Kan-Mitchell et al., In: Leukocyte Typing VI, T. Kishimoto et al., New York, 1997). MLV based vectors have been used to transduce CD34+ hematopoietic progenitor cells which were then differentiated into DC after weeks of in vitro culture. These DC were able to generated a specific T-cell mediated antitumor immune response in vitro (Henderson et al., Cancer Res. 56:3763, 1996; Reeves et al., Cancer Res. 56:56721996), although their relationship to naturally occurring DC is unknown.

Recent evidence suggest that DC are potent physiological adjuvants for induction of prophylactic or therapeutic antitumor immunity. In mice, DC pulsed with short synthetic peptides in vitro elicited protective immunity mediated by tumor specific CD4+ helper or CD8+ cytotoxic T cells (Nair et al., Int. J. Cancer 70:706, 1977) in vivo. Therapeutic efficacy was suggested by results of a pilot study in which lymphoma patients treated with autologous DC from the blood pulsed ex vivo with the lymphoma idiotype; patients produced antibodies and experienced clinical responses (Lynch et al., Nature Med. 3:625, 1997).

Although recent developments in combination drug therapy have had a tremendous impact on the treatment of AIDS patients in developed countries, the AIDS epidemic continues apace in its global devastation. The most effective means to curtail the spread of this disease would be to develop a safe and efficacious vaccine. One of the major problems in AIDS vaccine development is the weak and transient immune response from currently available vaccines.

There is compelling evidence that HIV-specific cytotoxic T lymphocytes (CTLs) are central to controlling HIV infection from studies in patients (Rowland-Jones et al., Adv. Immunol. 65:277,1997). Strong CTL responses have been identified particularly in nonprogressive patients and at the sites of infection. CTL also inhibit virus replication in vitro, and react to most HIV gene products, predominantly including gag, pol, and env, and this reactivity has been mapped. CTL epitopes cluster together in regions of pol, but were more evenly distributed through gag. Most epitopes were identified based on the binding motif of the Class I antigen (Brander et al., Clin. Exp. Immunol. 101:107, 1995). HLA-A2 donors have been shown to recognized at least three epitopes on gag and two on pol, one of which is an immunodominant epitope in the active site of the reverse transcriptase. Spontaneous response to pol, which should be a valuable target for immunotherapy, were rarely observed (McMichael and Walker, AIDS 8 (suppl. IZ): S155, 1994; Goulder et al., Nature Med. 3:212, 1997).

### SUMMARY OF THE INVENTION

The present invention is based on the discovery that lentivirus-transduced dendritic cells can be used as vaccines against HIV or other antigens. In a particular aspect, a human dendritic cell (DC)-based vaccine strategy was developed to induce virus-specific cytotoxic T cell (CTL) immunity.

In a first embodiment, the invention provides a method of inducing an immune response in a subject. The method includes administering to the subject, a therapeutically effective amount of a dendritic cell or a progenitor thereof, transduced with a replication defective pseudotyped lentiviral vector having a nucleic acid sequence encoding an antigen such that the antigen is presented on the surface of the dendritic cell.

In another embodiment, the invention provides a method of inducing an immune response in a subject including transducing a dendritic cell or a progenitor of a dendritic cell with a pseudotyped lentiviral vector comprising a nucleic acid sequence encoding an antigen such that the antigen is presented on the surface of the dendritic cell to produce a transduced dendritic cell and contacting the transduced dendritic cells with a T cell to produce an activated T cell, wherein at least one of the pseudotyped lentiviral vector, the transduced dendritic cell and the T cell, are administered to the subject.

In yet another embodiment, the invention provides a method of activating a T cell comprising contacting a T cell with a dendritic cell having an antigen on its surface, wherein the dendritic cell includes a pseudotyped lentiviral vector having a nucleic acid sequence encoding the antigen, wherein the contacting results in activating the T cell.

### BRIEF DESCRIPTION OF THE DRAWINGS

FIG. 1 is a schematic illustration of an HIV-1 provirus, and env-deleted HIV-1 vector encoding GFP, Env-encoding plasmids, a murine leukemia virus (MLV) vector encoding GFP, and a MLV package plasmid.

FIG. 2 is a plot of two color flow cytometric analysis of the expression of GFP in CD34+ cells delivered by an HIV-1 vector.

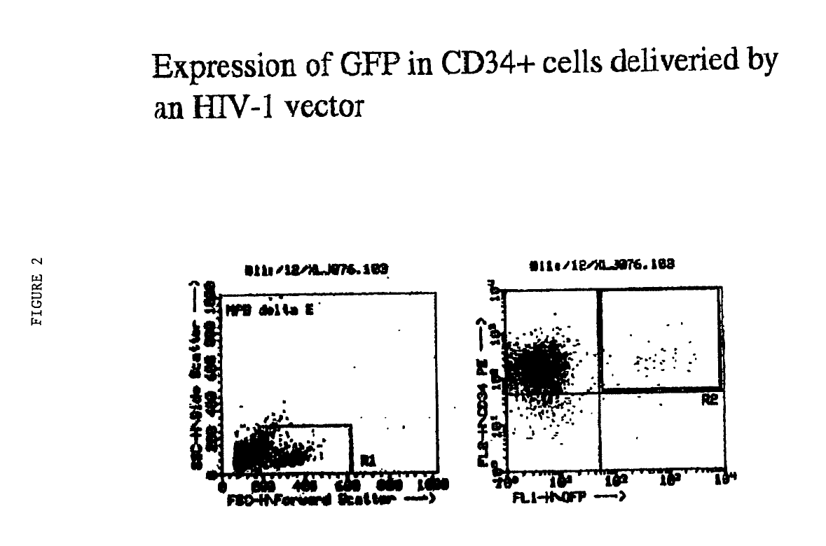

### DESCRIPTION OF THE PREFERRED EMBODIMENTS

The present invention provides a new method for inducing an immune response in a subject by administering a dendritic cell or a progenitor of a dendritic cell transduced with a pseudotyped lentiviral vector containing a nucleic acid sequence of interest such that the nucleic acid sequence of interest is expressed. In a particular example, the invention shows that HIV antigens were stably introduced into human DC by HIV-1 vectors pseudotyped with the VSV-G protein, which allows highly efficient transduction into the CD34+ progenitor cells as well as adherent moncytes (mo-DCs). The data show that HIV-1 vectors encoding HIV-1 antigens and a reporter gene successfully transduces CD34+ cells and mo-DC with high efficiency relative to murine retroviral vectors.

A “dendritic cell” is a bone marrow derived leukocyte which is an antigen presenting cell. Dendritic cells are able to prime naive T cells. In vivo, DC have been shown to present antigen to, and activate native CD4 T cells (Levin et al., J Immunol. 151: 6742-6750, 1993). Several populations of human DC have been identified. These include myeloid DC which can be produced from precursors after in vitro culture with granulocyte macrophage colony stimulating factor (GM-CSF) and interleukin-4 (IL-4). Dendritic cells can be generated from highly purified, adherent monocytes (mo-DC) cultured in GM-CSF and IL-4 (Kan-Mitchell et al., In: Leukocyte Typing VI, T. Kishimoto et al. (eds), New York, 1997, herein incorporated by reference). Another form of dendritic cells are low density APC (LDC) can be found in fresh mobilized peripheral blood monocytes (PBMC) that appear to function as mature APC, including an allogeneic mixed lymphocyte reaction (MLR). Fresh LDC express low levels of the monocyte marker CD24, and high levels of HLA-DR, and costimulatory molecules CD40, CD80, and CD86. Freshly isolated dendritic cells, primary cultures of dendritic cells, or dendritic cell lines can be utilized with the subject invention.

The dendritic cells used in the methods of the invention may be xenogeneic, allogeneic, syngeneic or autologous. Steinberg et al. (WO 93/20185) have disclosed methods for isolating primary dendritic cells and their precursors from tissue. Granucci et al., WO 94/28113, and Paglia et al. (J. Exp. Med 178:1893-1901, 1993) have disclosed dendritic cell lines isolated from primary cultures and then immortalized. McKay et al. (U.S. Pat. No. 5,648,219) have described immortalized dendritic cell lines. Dendritic cells can be dividing or nondividing. The phase “nondividing” cell refers to a cell that does not go through mitosis. Nondividing cells may be blocked at any point in the cell cycle (e.g., G₀/G₁, G₁/S, G₂/M), as long as the cell is not actively dividing. Preferably, primary cultures of autologous dendritic cells are used in the in vitro methods of the invention.

A “dendritic cell progenitor” is a cell which can ultimately give rise to dendritic cells following appropriate signaling. Dendritic cell progenitors express CD34. Procedures for purifying CD34⁺ cells have been described (Lane, TA, et al., Blood 85:275, 1985). An “immature dendritic cell” is a dendritic cell that expresses low levels of MHC class II, but is capable of endocytosing antigenic proteins and processing them for presentation in a complex with MHC class II molecules. These cells may be stimulated to become activated dendritic cells. An “activated dendritic cell” is a more mature dendritic cell that expresses class I and high levels of MHC class II, adhesion molecules such as ICAM-1, and costimulatory molecules such as B7-2. An activated dendritic cell is capable of endocytosing antigenic peptides and processing them for presentation.

The dendritic cells may be substantially enriched. An “substantially enriched” DC population refers to a substantially homogeneous population of antigen presenting cells (APCs) which are substantially free from other cells with which they are naturally associated. In general, a substantially enriched population of selected cells is a population wherein the majority of, or at least about 90% of the cells, are the selected cell type. For example, enriched dendritic cells contain about 10% or less fibroblasts or other immune cells and most preferably contain about 5% or less of such cells. An enriched population of APCs can be achieved by several methods known in the art. For example, and enriched population of cells can be obtained using immunoaffinity chromatography using monoclonal antibodies specific for determinants found only on DCs.

Enriched populations can also be obtained from mixed cell suspensions by positive selection (collecting only DCs), or negative selection (removing cells which are not DCs). The technology for capturing specific cells on affinity materials is well known in the art (Wigzed, et al., J. Exp. Med. 129:23, 1969; Wysocki et al., Proc. Natl. Acad. Aci. USA 75:2844. 1978; Schrempf-Decker et al., J. Immunol Meth. 32:285, 1980; Muller-Sieberg et al., Cell 44:653, 1986). Monoclonal antibodies against antigens specific for mature, differentiated cells have been used in a variety of negative selection strategies to remove undesired cells, for example to deplete T cell or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin et al., Blood 63:904, 1984). Enriched DC composition can be obtained from a mixture of lymphocytes, since dendritic cells lack surface immunoglobulin (e.g., IgG) or T cell markers, and do not respond to B or T cell mitogens in vitro. DC also fail to react with MAC-1 monoclonal antibody, which reacts with all macrophages. Therefore, MAC-1 provides a means of negative selection that can be used in order to produce a substantially enriched population of DC.

Procedures for separation of cells may include magnetic separation, using antibody-coated magnetic beads, affinity chromatography, cytotoxic agents joined to a monoclonal antibody or used in conjunction with a monoclonal antibody, for example, complement and cytotoxins, and “panning” with antibody attached to a solid matrix, for example a plate or another convenient technique. Techniques providing accurate separation include fluorescence cell sorters which may have a plurality of color channels, low angle, and obtuse light scattering detecting channels, impedance channels, amongst others.

In the method of the invention, dendritic cells or progenitors of dendritic cells can be transduced with an effective amount of a pseudotyped lentiviral vector containing a nucleic acid sequence which encodes an antigen. The nucleic acid sequence can then be transcribed and translated by the dendritic cell to produce the antigen. The antigen can, therefore, be presented on the surface of the dendritic cell.

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase. Preferably, the recombinant retrovirus used in the method of the invention is lentivirus-derived such as a recombinant lentivirus that is a derivative of human immunodeficiency virus (HIV) or a recombinant lentivirus that is a derivative of feline immunodeficiency virus (FIV) . The retrovirus is replication-defective, such that assembly into infectious virions only occurs in the presence of an appropriate helper virus or in a cell line containing appropriate sequences enabling encapsidation.

Recombinant retrovirus (e.g., lentivirus) produced by standard methods in the art can be replication-defective, and require assistance in order to produce infectious vector particles. Typically assistance is provided, for example, by using a helper cell line that provides the missing viral functions. The helper cell lines include plasmids that are missing a nucleotide sequence which enables the packaging mechanism to recognize an RNA transcript for encapsidation. Helper cell lines which have deletions of the packaging signal include, but are not limited to, Ψ2, PA317 and PA12, for example. Suitable cell lines produce empty virions, since no genome is packaged. If a retroviral vector is introduced into such cells in which the packaging signal is intact, but the structural genes are replaced by other genes of interest, the vector can be packaged and vector virion produced.

The retroviral genome and the proviral DNA have three genes: the gag, the pol, and the env, which is flanked by two long terminal repeat (LTR) sequences. The gag gene encodes the internal structural (matrix, capsid, and nucleocapsid) proteins; the pol gene encodes the RNA directed DNA polymerase (reverse transcriptase), and the env gene encodes viral envelope glycoproteins. The 5' and 3' LTRs serve to promote transcription and polyadenylation of the virion RNAs. The LTR contains all other cis-acting sequences necessary for viral replication. Lentiviruses have additional genes including vif, vpr, tat, rev, vpu, nef, and vpx (in HIV-1, HIV-2, FIV and/or SIV).

Adjacent to the 5' LTR are sequences necessary for reverse transcription of the genome (the tRNA primer binding site) and for efficient encapsidation of viral RNA into particles (the Psi (Ψ) site). If the sequences necessary for encapsidation (or packaging of retroviral RNA into infectious virions) are missing from the viral genome, the result is a cis defect which prevents encapsidation of genomic RNA. However the resulting mutant is still capable of directing the synthesis of all virion proteins.

The retroviruses (e.g., lentivirus) of use with the subject invention have been genetically modified such that the structural, infectious genes of the native virus have been removed and replaced with other nucleic acid sequences. Thus the virus is replication-defective, although it can still contains the encapsidation signal and thus can be packaged into virions. After infection of a dendritic cell by the recombinant retrovirus, the virus injects its nucleic acid into the cell and the retroviral genetic material can integrate into the host dendritic cell's genome. The transferred retrovirus genetic material is then transcribed and translated into proteins which can be expressed on the surface of the dendritic cell.

The recombinant retrovirus (e.g., lentivirus) of the subject invention is a “pseudotyped” retrovirus, which indicates that the envelope of the retrovirus has been replaced by the envelope of another virus. The envelope can be derived from any virus, including retroviruses. In addition, the envelope can be amphotropic, xenotropic or ecotropic, for example. The envelope may be an amphotropic envelope protein (e.g., MLV) which allows transduction of cells of human and other species, or may be ecotropic envelope protein, which is able to transduce mouse and rat cells. The envelope gene is not contained within the lentiviral genome of the nucleic acid vector, but rather is provided in the packaging system used to generate the recombinant vector (e.g., transient co-transfection or stable, inducible cell lines) to produce a recombinant pseudotyped lentivirus or virion for transduction of DCs. Packaging cell lines will be known to those of skill in the art.

Examples of viral envelope proteins useful for pseudotyping a vector used in the methods of the invention include, but are not limited to, Molony murine leukemia virus (MoMuLV), Harvey murine sarcoma virus (HaMuSV), murine mammary tumor virus (MMTV), gibbon ape leukemia virus (GaLV), human immunodeficiency virus (HIV), feline immunodeficiency virus (FIV), Rous Sarcoma Virus (RSV), and Vesicular Stomatis Virus (VSV) protein G. In an exemplary lentiviral vector described herein, the VSV-G envelope is utilized. Further, exemplary lentiviral vectors for use in the methods described herein, are provided in co-pending U.S. patent application Ser. No. 08/936,633, filed Sep. 24, 1997, which is herein incorporated by reference in its entirety.

It may also be desirable to target the virus by linkage of the envelope protein with an antibody or a particular ligand for targeting to a receptor of a particular cell type. By inserting a sequence (including a regulatory region) of interest into the viral vector, if the ligand for the receptor is present on a specific target cell, for example, the vector is now specific for the target cell. The retroviral vectors of use with the subject invention can be made target specific by inserting for example, a glycolipid, or a protein. For targeting to dendritic cells, a sequence of particular interest is specific for CD86. CD86 (B7-2) is expressed at high levels on DC, but is generally absent on nonantigen-presenting cells. By incorporating a binding domain for CD86 in the coat protein of the retrovirus, genes are delivered specifically to DC. CD86 binding domains include its counter-receptors CTLA-4 and CD28, and antibodies that specifically bind CD86. For example, the nucleotide sequence encoding the binding domain of CTLA-4 is isolated by conventional technology, for example through the use of restriction endonucleases, PCR amplification, etc., and inserted into an appropriate retroviral envelope protein, such as VSV-G. Those of skill in the art will know of, or can readily ascertain without undue experimentation, other methods to achieve delivery of a retroviral vector to a target cell.

Several cis-acting viral sequences are necessary of the viral life cycle. Such sequences include the T packaging sequence, reverse transcription signals, integration signals, viral promoter, enhancer, and polyadenylation sequences. The vector contains at least one cloning site for a nucleic acid sequence encoding an antigen which is to be transferred to the dendritic cell. The nucleic acid sequence inserted into this site is a sequence encoding an antigen. An “antigen” is any polypeptide or fragment thereof, that can be recognized by a cell of the immune system or by an antibody. This antigen may be a “heterologous” nucleic acid sequence, which refers to a sequence which originates form a foreign species, or if from the same species, it may be substantially modified from the original form. Alternatively, the nucleic acid sequence encoding an antigen may encode an antigen from the same species. The nucleic acid sequence encoding an antigen may also encode a selectable marker gene. Marker genes can be utilized to assay for the presence of the vector. Typical selection genes encode proteins that confer resistance to antibiotics and other toxic substrates, e.g., histidinol, puromycin, hygromycin, neomycin, methotrexate, etc. Selectable makers also include proteins which can be assayed by physical means, such as fluorescence or an enzymatic reaction. Examples of such markers include, but are not limited to, β-galactosidase, luciferase, or green fluorescent protein.

The nucleic acid encoding an antigen can encode a viral antigen. It is advantageous to select viral antigens which are less likely to mutate during the course of viral infection for presentation in potent antigen presenting cells, namely dendritic cells. In one embodiment the viral antigen is a lentiviral antigen. The lentiviral antigen can include, but is not limited to, the gag, pol, env, env, vpr, vif, nef, vpx, tat, rev, vpu gene products, or immunogenic fragments thereof. One nonlimiting example of the use of nucleic acid encoding a gag protein of HIV-1. This would allow the antigens to be presented exclusively to the immune system with multiple, presumably optimal, immunostimulatory signals to amplify many different T-dependent responses, including both proliferative and cytotoxic responses on CD4+ and CD8+ T cells. The nucleic acid of interest can encode a fusion peptide. A “fusion peptide” is a combination of two or more antigenic peptides that are linked together.

The nucleic acid encoding an antigen can encode a tumor specific antigen. Tumors can express “tumor antigens” which are antigens that can potentially stimulate apparently tumor-specific immune responses. These antigens can be encoded by normal genes and fall into several categories (1) normally silent genes, (2) differentiation antigens (3) embryonic and fetal antigens, and (4) clonal antigens, which are expressed only on a few normal cells such as the cells from which the tumor originated. Tumor-specific antigens can be encoded by mutant cellular genes, such as oncogenes (e.g., activated ras oncogene), suppressor genes (e.g., mutant p53), fusion proteins resulting from internal deletions or chromosomal translocations. Tumor-specific antigens can also be encoded by viral genes, such as RNA or DNA tumor viruses. In the treatment of lymphoma, the idiotype of the secreted immunoglobulin serves as a highly specific tumor associated antigen. By “idiotype” is meant the collection of V-region determinants specific to a specific antibody or a limited set of antibodies. The nucleic acid encoding an antigen can encode a lymphoma specific idiotype. For use with tumor antigens, one might prefer to use a non-HIV based vector for public policy reasons.

The nucleic acid encoding an antigen is operably linked to a regulatory nucleic acid sequence. The term “operably linked” refers to functional linkage between the regulatory sequence and the nucleic acid encoding an antigen. Preferably, the nucleic acid encoding an antigen is operably linked to a promoter, resulting in a chimeric gene. The nucleic acid encoding an antigen is preferably under control of either the viral LTR promoter-enhancer signals or of an internal promoter, and retained signals within the retroviral LTR can still bring about efficient integration of the vector into the genome of the DC.

The promoter sequence may be homologous or heterologous to the nucleic acid encoding an antigen. A wide range of promoters may be utilized, including viral or mammalian promoters. Cell or tissue specific promoters can also be utilized, such as the CD86 promoter. Suitable mammalian and viral promoters of use in the method of the invention are available in the art.

The pseudotyped lentiviral vector of use in the invention can further comprise nucleic acid encoding a cytokine. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines are known to influence the maturation of dendritic cells and to be involved in the immune response to an antigen. In one embodiment, the pseudotyped lentiviral vector further comprises a cytokine which is involved in the maturation of dendritic cells. Examples of cytokines include, but are not limited to, interleukin-4 (IL-4), interleukin-2 (IL-2), interleukin-3 (IL-3), granulocyte macrophage colony stimulating factor (GM-CSF), stem cell factor (SCF), and the Flt-3/Flk-2 ligand (FL). The nucleic acid encoding a cytokine is operably linked to a regulatory nucleic acid sequence, such a promoter. The promoter sequence may be homologous or heterologous to the nucleic acid encoding a cytokine. The nucleic acid encoding a cytokine is preferably under control of either the viral LTR promoter-enhancer signals or of an internal promoter. A wide range of promoters can be used, such as viral and mammalian promoters, and are available in the art.

The lentiviral vector of use with the invention is capable of transferring the nucleic acid encoding an antigen into a dendritic cell such that the antigen is expressed by the dendritic cell. The term “nucleic acid” refers to any nucleic acid molecule, preferably DNA. The nucleic acid may be derived form a variety of sources including DNA, cDNA, synthetic DNA, RNA, or combinations thereof. Such nucleic acid sequences may comprise genomic DNA which may or may not include naturally occurring introns. Moreover, the genomic DNA may be obtained in association with promoter regions, introns, or poly A sequences. Genomic DNA may be extracted and purified from suitable cells by means well known in the art. Alternatively messenger RNA (mRNA) can be isolated. The mRNA can be used to produce cDNA by reverse transcription or other means.

By “transduction” or “transformation” is meant a genetic change induced in a cell following incorporation of new DNA (i. e., DNA exogenous to the cell). The new DNA can be present in the cell as an extrachromosomal or chromosomally integrated element. Where the cell is a mammalian cell, the genetic change is generally achieved by introduction of the DNA into the genome of the cell (i. e., stable). Transduction can take place either in vivo or in vitro. The retroviral vectors of use with the subject invention can be used to transduce dendritic cells either in vivo or in vitro by methods well known to one of skill in the art.

Expression of the nucleic acid of interest occurs as a result of the pseudotyped lentiviral vector entering the dendritic cell. By “expression” is meant the production or a change in level of either mRNA or polypeptide of the nucleic acid of interest. Expression of a nucleic acid of interest in a dendritic cell or a progenitor of a dendritic cell can result in presentation of the nucleic acid of interest. “Presentation” is binding of a peptide or a fragment of a peptide encoded by the nucleic acid of interest to class I or class II MHC molecules to form a bimolecular complex recognized by T cells. This complex is then transported to, and displayed on, the surface of the dendritic cell. Activation of the dendritic cell can further be manifested by the expression of (1) adhesion molecules that promote the physical interaction between T cells and dendritic cells, (2) membrane bound growth or differentiation molecules (costimulators) that promote T cell activation, and (3) soluble cytokines, such as IL-1 and TNF. A “transduced dendritic cell” is a dendritic cell that has been transduced with a pseudotyped lentiviral vector containing a nucleic acid sequence encoding an antigen, such that the nucleic acid is expressed and the antigen is presented to the immune system.

The transduced dendritic cell comes in contact with a T cell to produce an activated T cell. By “contacting” is meant allowing the dendritic cell and the T cell to interact in suitable conditions, such that the T cell is activated. Contacting can occur either in vivo or in vitro. In one embodiment, the dendritic cell is transduced in vitro (e.g., ex vivo), and contacted with a T cell in vivo. In another embodiment, the contact of the transduced dendritic cell with the T cell is performed in vitro (Henderson et al., 1996, supra; Reeves et al., 1996, supra, herein incorporated by reference). In this embodiment, the T cells are first isolated. Methods for isolating T cells are well known in the art. T cells are isolated from an autologous or allogeneic donor by flow cytometry, panning, antibody-magnetic bead conjugates, etc., as known in the art, or a T cell line may be employed. The cells may be transfected with an expression vector that encodes a protein domain containing addressing information for cell type specificity, e.g., a ligand for a receptor expressed by activated T cells; a counter-receptor for addressins, selectins etc.

The dendritic cell and the T cell interact under conditions where the T cell can be activated. T cell activation occurs when a polypeptide is presented on an antigen presenting cell, such as a dendritic cell, in the context of MHC class I or class II. A T cell expressing T cell receptor-CD3 complex then undergoes molecular events which indicate the stimulation of the T cell. Molecular events which indicate T cell activation include, but are not limited to, the activation of a src-family tyrosine kinase, phosphorylation of phospholipase C, or the secretion of cytokines, such as IL-2. Culture requirements for T cell activation in vitro are well known in the art (Henderson et al., 1996, supra; Reeves et al., 1996, supra).

In one embodiment of the invention, a therapeutically effective amount of a dendritic cell or a progenitor of a dendritic cell transduced with an effective amount of a pseudotyped lentiviral vector containing a nucleic acid sequence encoding an antigen of interest is administered to a subject. In another embodiment, at least one of (1) the pseudotyped lentiviral vector containing a nucleic acid sequence encoding an antigen, (2) a dendritic cell transduced by the lentiviral vector, and (3) a T cell activated by the transduced dendritic cell, are administered to a subject. By subject is meant any mammal, preferably a human.

By “therapeutically effective amount” is meant a sufficient amount to stimulate either a humoral or cellular immune response. The term “immune response” refers herein to a T cell response or to B cell response resulting in increased serum levels of antibodies to an antigen, or to the presence of neutralizing antibodies to an antigen. The term “protection” or “protective immunity” refers herein to the ability of the serum antibodies and the T cell response induced during immunization to protect (partially or totally) against disease caused by an agent. Preferably, the immune response is a cellular response. Most preferably, the immune response is a cytotoxic T cell (CTL) response.

In one embodiment, the method of the invention can be used to stimulate the immune response in a virally-infected subject (e.g., stimulating the immune response in a subject infected with HIV). In another embodiment, the method of the invention can be used to protect against a viral infection, by stimulating the immune response against the virus. In yet another embodiment, the method of the invention can be used to stimulate an immune response against a neoplasm. In a further embodiment, the method of the invention can be used stimulate the immune response in order to protect against metastases of a tumor.

Tumors are antigenic and can be sensitive to immunological destruction. The term “tumor” is usually equated with neoplasm, which literally means “new growth”. A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors (called “cancer”) are almost never encapsulated but invade adjacent tissue by infiltrative destructive growth. This infiltrative growth can be followed by tumor cells implanting at sites discontinuous with the original tumor. The method of the invention can be used to stimulate an immune response directed against neoplastic disorders, including but not limited to: sarcoma, carcinoma, fibroma, lymphoma, melanoma, neuroblastoma, retinoblastoma, and glioma.

“Administering” the retroviral vectors, dendritic cells, or activated T cells of use in the present invention may be accomplished by any means known to the skilled artisan. The retrovirus (e.g., lentivirus) can be administered to a patient as packaged virus particles, or in the provirus form, i.e., integrated DNA in dendritic cells.

According to one method of the invention, the pseudotyped lentiviral vector comprising a nucleic acid encoding an antigen is replication-defective, and can be packaged in vitro (see above). The packaged virus can then be delivered to the subject in order to transduce the dendritic cells of the subject. The pseudotyped lentiviral vector comprising a nucleic acid encoding an antigen can be delivered in combination with dendritic cells transduced with the same or another pseudotyped lentiviral vector comprising a nucleic acid encoding an antigen. The pseudotyped lentiviral vector comprising a nucleic acid encoding an antigen can be also be delivered in combination with T cells activated by dendritic cells transduced with the same or another pseudotyped lentiviral vector comprising a nucleic acid encoding an antigen.

The clinical administration of retroviruses has been accomplished by the by the direct injection of virus into tissue, and by the administration of the retroviral producer cells. Methods for delivering retrovirus and retroviral producer cells to a subject are well known in the art, and include, but are not limited to, intramuscular, intravenous, intraperitoneal, and subcutaneous delivery. The pseudotyped lentivirus comprising a nucleic acid sequence encoding an antigen may be prepared as formulations at a pharmacologically effective dose in pharmaceutically acceptable media, for example normal saline, PBS, etc. The additives may include bactericidal agents, stabilizers, buffers, adjuvants, or the like. The virus may be administered as a cocktail, or as a single agent.

The dosage of the therapeutic formulation will vary widely, depending upon the nature of the disease, the frequency of administration, the manner of administration, the clearance of the agent from the host, and the like. The dose may be administered as infrequently as weekly or biweekly, or fractionated into smaller doses and administered daily, semiweekly, etc. to maintain an effective dosage level. The formulation will be administered at a dosage sufficient to induce an immune response. The determination of dosage will vary with the condition that is being treated. Useful measures of inflammatory activity are the release of proinflammatory cytokines, e.g., IL-2, IFN-γ, TNFα, enhanced populations of activated T cells at disease associated sites, other measures of T cell activity, and measure of B cell activity and the production of antibodies, as known in the art.

In a method of the invention, dendritic cells transduced with a pseudotyped lentiviral vector containing a nucleic acid encoding an antigen are delivered to the subject. Transduction of the dendritic cell is performed in vitro, generally with isolated cell populations or cell lines, using culture methods for dendritic cells or dendritic cell progenitors (see above). Dendritic cells may be xenogeneic, allogeneic, syngeneic or autologous, preferably autologous, in order to reduce adverse immune responses. Dendritic cells can localize to the site for treatment after administration to a host animal. The dendritic cells may be administered in any physiologically acceptable medium, normally intravascularly, although they may also be introduced into lymph node or other convenient site, where the cells may find an appropriate site for expansion and differentiation. Any of the transplantation or implantation procedures known in the art can be utilized. For example, the selected cells or cells of interest can be surgically implanted into the recipient or subject. Further, the cells can be administered in an encapsulated form or non-encapsulated form. Preferably the cells are nonencapsulated.

Transplantation or implantation is typically by simple injection through a hypodermic needle having a bore diameter sufficient to permit passage of a suspension of cells without damaging the cells or tissue coating. For implantation, the typically the cells are formulated as pharmaceutical compositions together with a pharmaceutically-acceptable carrier. Such compositions contain a sufficient number of cells which can be injected into, or administered through a laparoscope to, a subject, usually into the peritoneal cavity. However, other transplantation sites can be selected depending upon the specific dendritic cells and desired biological effect; these sites include the thymus, liver, spleen, kidney capsule, lymph node, and the like. Usually, at least 1×10⁵ cells will be administered, preferably 1×10⁶ or more. The cells may be frozen at liquid nitrogen temperatures and stored for long periods of time, being capable of use on thawing. Once thawed, the cells may be expanded.

The dendritic cells also can be encapsulated prior to transplantation. Although the cells are typically microencapsulated, they can be encased in various types of hollow fibers or in a block of encapsulating material. A variety of microencapsulation methods and compositions are known in the art. A number of microencapsulation methods for use in transplant therapy have focused on the use of alginate polymers or agarose to supply the encapsulation compositions. Alginates are linear polymers of mannuronic and guluronic acid residues which are arranged in blocks of several adjacent guluronic acid residues forming guluronate blocks and block of adjacent mannuronic acid residues forming mannuronate blocks, interspersed with mixed, or heterogenous blocks of alternating guluronic and mannuronic acid residues. Generally, monovalent cation alginate salts are soluble, e.g., Na-alginate.

Divalent cations, such as Ca⁺⁺, Ba^{+ +}or Sr⁺⁺, tend to interact with guluronate, and the cooperative binding of these cations within the guluronate blocks provides the primary intramolecular crosslinking responsible for formation of stable ion-paired alginate gels. Alginate encapsulation methods generally take advantage of the gelling of alginate in the presence of these divalent cation solutions. In particular, these methods involve the suspension of the material to be encapsulated, in a solution of monovalent cation alginate salt, e g., sodium. Droplets of the solution are then generated in air and collected in a solution of divalent cations, e.g., CaCl₂. The divalent cations interact with the alginate at the phase transition between the droplet and the divalent cation solution resulting in the formation of a stable alginate gel matrix being formed. Generation of alginate droplets has previously been carried out by a number of methods. For example, droplets have been generated by extrusion of alginate through a tube by gravitational flow, into a solution of divalent cations. Similarly, electrostatic droplet generators which rely on the generation of an electrostatic differential between the alginate solution and the divalent cation solution have been described. The electrostatic differential results in the alginate solution being drawn through a tube, into the solution of divalent cations. For a general discussion of droplet generation in encapsulation processes, see, e.g., M. F. A. Goosen, Fundamentals of Animal Cell Encapsulation and Immobilization, Ch. 6, pp. 114-142 (CRC Press, 1993).

Further, methods have been described wherein droplets are generated from a stream of the alginate solution using a laminar air flow extrusion device. Specifically, this device comprises a capillary tube within an outer sleeve. Air is driven through the outer sleeve and the polymer solution is flow-regulated through the inner tube. The air flow from the outer sleeve breaks up the fluid flowing from the capillary tube into small droplets. See U.S. Pat. No. 5,286,495. Viable tissue and cells have been successfully immobilized in alginate capsules coated with polylysine. See J. Pharm. Sci. 70:351-354 (1981). The use of these coated capsules in pancreatic islet transplantation to correct the diabetic state of diabetic animals has been described (Science 210:908-909 (1981)).

In another embodiment, dendritic cells are used to activate T cells in vitro, as described above, and the activated T cells are then introduced into a subject. The adoptive transfer of immune cells is well known in the art (e.g., Rohane et al. (1995) Diabetes 44:550-554). The T cells can also be administered using the methods described above for delivering dendritic cells. The activated T cells may be administered in any physiologically acceptable medium, normally intravascularly, although they may also be introduced into lymph node or other appropriate site, such as the site of a neoplasm. Any of the transplantation or implantation procedures known in the art can be utilized. For example, the T cells can be surgically implanted into the recipient or subject. The activated T cells can be administered alone, or can be administered in conjunction with a pseudotyped lentiviral vector containing a nucleic acid sequence encoding an antigen and/or a dendritic cell transduced with the lentiviral vector.

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLE 1

### Efficient Transduction of CD34+Cells With a VSV-G Pseudotyped HIV-1 Vector

Mobilized peripheral blood was obtained from normal donors with informed consent and Institutional Review Board approval. The procedure for purifying CD34+ cells has been described previously (Lane, T. A., et al., Blood 85:275, 1995, herein incorporated by reference).

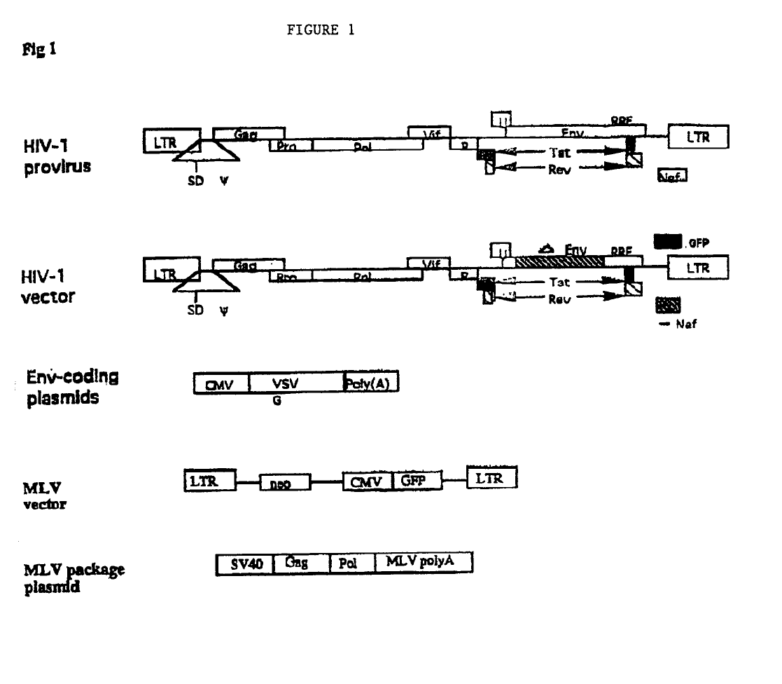

VSV-G pseudotyped HIV-1 vectors were prepared by cotransfecting COS cells by electroporation with plasmids expressing VSV-G and an envelope-defective HIV-1 genome expressing the GFP gene (FIG. 1). A similar MLV-based, VSV-G pseudotyped retroviral vector was similarly prepared (FIG. 1). Cell culture supernatants were collected at 72 h posttransfection and titered on Hela cells by assaying for GFP expression.

CD34+ cells (10⁶/ml) were transduced with HIV-1 and MLV vectors at MOI's of 0.5 to 1 in the presence of recombinant human cytokines (GM-CSF 10 ng/ml; SCF 40 ng/ml; and IL-3 10 ng/ml) and 4 μg/ml protamine sulfate. The cells were transduced for 1 to 2 hr at 26 to 28° C. while centrifuging at 2400×g, washed 5 times with IMDM containing 10% FCS after 24 hr. Cells were cultured for another 24 hr before FACs analysis and methylcellulose colony assays.

DNA was extracted from CD34+ population after transduction. DNA was amplified using GFP specific primers in conditions recommended by the manufacturer. The PCR was done by 94° C. for 2′ followed by 30 cycles of 94° C. for 30″. 56° C. for 30″ and 72° C. for 1 min. DNA products were analysis on 1% agarose gel and visualized by UV.

The infection efficiencies of HIV-1 and MLV vectors pseudotyped with VSV-G proteins of CD34+ cells obtained from mobilized peripheral blood of normal donors was compared. The multiplicity of infection was kept the same by using the same titer obtained by GFP expression in Hela cells. HIV-1 vector packaged in VSV-G showed a five- to tenfold greater transduction of CD34+ cells compared with that for the VSV-G pseudotyped MLV retroviral vectors as measured by DNA PCR. Furthermore, the HIV-1 vector induced threefold higher expression level of GFP as indicated by FACs analysis of the transduced cells.

### EXAMPLE 2

### Generation of Multilineage Progeny Cells From Transduced Progenitor Cells

To determine if multipotential hematopoietic progenitor cells had been transduced by the HIV-GFP vector (see Example 1) and to ascertain the fate of GFP expression in lineage-committed cells, colonies of granulocytes-macrophages (CFU-GM) and colonies of erythrocytes (CFU-e) derived from the sorted CD34+ cells expressing GFP were assayed by immunomicroscopy.

CD34+ cells expressing GFP were plated at 2×10⁵ cells/ml in IMDM supplemented with 10% FCS, penicillin/streptomycin (100 nits/ml). The cells were cultured in the methylcellulose plates (Stem Cell Technology) in the presence of combination of the cytokines mentioned above plus IL-6 (50 ng/ml) and Epo 2-3 units/ml for myeloid cell differentiation. For differentiation of DCs, TNF-a (100 units/ml), GM-CSF (10 ng/ml, SCF (40 ng/ml), and IL4 (400 units/ml) were added in the media. These cytokines were added to the cultures every 48 hr and the cells expanded as necessary for the growth of DCs.

For fluorescence microscopy, the cells growing at day 14 in the presence of cytokines for DCs differentiation were stained as above using either antibody CD1a-PE, or antibody CD14a-PE. After staining, the cells were washed and resuspended at 10⁵ cells/ml. Approximately 10⁴ cells were applied to standard glass microscope slides, and observed using a Nikon FXA photomicroscope. Colonies were collected at day 14 postinfection to detect GFP expression. The results showed that about 40 to 60% of cells in each type of colony assayed expressed GPF, indicating that the progenitor cells were stably transduced and maintained high levels of gene expression from the HIV-1 LTR. To test whether the expression of HIV viral proteins could interfere with the differentiation capacity of CD34+, transduced CD34+ cells were sorted according to the GFP-expression. GFP+CD34+ cells were plated on methylcellulose plates. In comparison with nontransduced CD34+ cells, the numbers of colonies for CFU-GM and CFU-e were decreased about 10 to 20% (Table 1). Immunofluorescence microscopy showed that high levels of HIV-1 expression led to apoptosis of the progeny cells, which may account for 10 to 20% loss of the cells.

TABLE 1
The Effect of HIV-1 Transduction on Colony Formation of CD34 + Cells

| Vectors | CFU-GM | BFU-e |
|---|---|---|
| LNL | 135 | 112 |
| MLV (control) | 156 | 125 |
| HIV-1 | 128 | 104 |
| HIV-1 | 121 | 102 |
| HIV-1 | 108 | 89 |
| HIV-1 | 105 | 92 |

Several different cytokine combinations have been reported to induce differentiation of DCs from precursor CD34+ cells in peripheral blood. Cytokine combinations which gave about 50 to 60% DCs were chosen (Henderson, Cancer Res. 56:3763, 1996). This combination allowed the maximal proliferation of DCs while retaining the CD1a^{bright}CD14-phenotype. To generate DCs that express GFP, CD34+ cells were purified from mobilized peripheral blood and transduced with HIV-1 vector. GPF expressing CD34+ were collected by cell sorting and used to differentiate into DCs in the presence of combination cytokines. DCs expressing GFP were identified by cell morphology such as cytoplasmic tails and by negative CD14 staining. About 50 to 70% of the culture repeatedly displayed typical DC morphology and expressing GFP during the 6 week culture period. Since fluorescence detection of GFP requires high levels of gene expression, DCs have sufficient transcriptional factors to ensure high level expression of genes from the HIV-1 LTR promoter. The high level of HIV-1 gene expression did not interfere with DC proliferation, in contrast to conclusion from a previous publication (Granelli-Piperno, A., et al., Proc. Natl. Acad Sci. USA 92:10944, 1995).

VSV-G pseudotyped HIV-1 vectors are efficient in transducing CD34+ cells with or without cytokine stimulation. In the studies described here, the feasibility of stable gene transfer into human DCs by HIV-1 vectors was demonstrated. The process of HIV transduction and expression of HIV genes does not alter or influence the generation or differentiation of DCs from CD34+ cells.

### EXAMPLE 3

### Mo-DC From Leukapheresis Samples

DC were generated from monocytes in mobilized PBMC of healthy donors and breast cancer patients. From 2×10 PBMC collected by leukapheresis, 10⁹ mo-DC were obtained. Mononuclear cells were twice purified by Ficoll-Hypaque gradient centrifugation and monocytes were isolated by adherence to plastic in RPMI alone overnight. Nonadherent cells were removed by vigorous washes and cultured for 7 d in RPMI with 10% fetal calf or human AB serum containing 100 ng/ml each of GM-CSF and IL-4. Within 3 d, the cells detached from the plastic and became a suspension culture. However, if the cells were replated onto a fresh tissue culture flask or glass, they reattached and became characteristically dendritic . Detailed analysis of over 15 mo-DC preparations from 10 healthy donors and 5 breast cancer patients revealed no significant different in surface CD phenotypes (Table 2).

TABLE 2
CD Phenotypes of mo-DC are Characteristic of Mature DC

| | |
|---|---|
| MHC antigens | HLA Class I++ and Class II++ |
| Costimulatory molecules | CD80+ and CD86++ |
| T cell markers | CD2−, CD3−, CD4−, CD8−, CD95+ |
| B cell markers | CD19− and CD83 |
| NK markers | CD16−, CD56− and CD57− |
| Myeloid markers | CD14−, CD13+, CD64− |
| Leucocyte markers | CD45RA−, CD45RO+, CD32+ |

In vitro Cytokine Requirements for Maturation of DC

Antigen presenting function of mo-DC was enhanced by adding IL-2, IL-3, SCF and FL to culture medium containing GM-CSF plus IL-4. Consistent with the prevailing idea that GM-CSF is not the major growth factor for DC in vivo, GM-CSF could be replaced by FL and SCF in the differentiation of monocytes to DC, although IL-4 must be present. There was no difference between the surface CD phenotypes of mo-DC derived from FL and SCF from GM-CSF, and they were all equally effective APC.

Low Density Mature DC Found Only in Mobilized PBMC

Putative novel DC precursors in 5 mobilized PBMC have been identified. A population of small (similar to a medium-sized lymphocyte), round, low density cells representing up to 5 to 10% of the total cell number was isolated over metrizimide gradients (Bender, A., et al., J. Immunol. Meth. 196:121, 1996). The cells were devoid of lineage markers for T, B and NK cells (CD3−, CD19−, CD16/56−) but were negative or DC ₁₄^{dim} and stained strongly for HLA-DR. Two samples were CD40−CD80−CD86−. As with monocytes, the fresh cells failed to elicit a MLR. After a 7 d culture in GM-CSF and IL-4, however, they became CD14−, CD40+, CD80+, CD86+ and HLA-DR+DC. These cultured low density DC were distinct from mo-DC derived from the same donor. They were smaller and had dense nuclei. Furthermore, when cultured in an additional third cytokine such as IL-2 and IL-3, they underwent dramatic morphological changes while mo-DC were not similarly affected. These data suggest that the low density cells may be a less differentiated precursor of DC than monocytes in the differentiation pathway of the myeloid DC.

Of great interest, the low density DC14+ HLA-DR+ cells in the three samples were found to constitutively express CD40, CD80 and CD86. The fresh cells were fully competent APC, inducing a MLR without further manipulation or exposure to cytokines. These, too, upon culture with GM-CSFR an IL-4 for 7 d became dendritic. The low density DC represented 5 to 10% of the cells in mobilized PBMC, a yield that is unprecedented for other known DC precursors. Thus, mobilized blood is an enriched and invaluable source for DC and their precursors.

### EXAMPLE 4

### Efficient Transduction and Transgene Expression of Mature Mo-DC With Pseudotyped HIV Vectors

A preparation of mo-DC, verified to be >95% homogeneous, was transduced with the HIV-1 vector in the presence of GM-CSF (100 ng/ml) and IL-4 (100 ng/ml) and 4 mg/ml polybrene. The cells were transduced at a MOI of three times for 30 min. at 25° C. while centrifuging at 2,400×g. Cells were washed five times with medium and cultured in RPMI 1640 containing 10% human AB serum, GM-CSF and IL-4. mo-DC were fixed with 4% paraformaldehyde in PBS at 2, 3, 4, 5 or 6 days after transduction. The percent of GFP-expressing mo-DC among unselected mo-DC was determined by counting under fluorescent microscopy. Greater than 40% of the cells expressed GFP at 4 days posttransduction.

TABLE 3
Transduction of Mature Mo-DC
With the VSV(G)-pseudotyped HIV-1 Vector

| Days After Transduction | % GFP Positivity |
|---|---|
| 2 | 11.6 |
| 3 | 33.6 |
| 4 | 43.7 |
| 5 | 33.7 |
| 6 | 31.0 |

In summary, HIV antigens have been stably introduced into human DC by HIV-1 vectors pseudotyped with the VSV-G protein, which allows highly efficient transduction into the CD34+ progenitor cells as well as mo-DC. The data showed that (1) HIV-1 vector encoding HIV-1 antigens and a GFP reporter gene successfully transduces CD34+ cells and mo-DC with high efficiency relative to murine retroviral vectors. (2) HIV-1 vector transduction does not interfere with CD34+ cells differentiation in vitro nor alters the morphology or surface CD phenotype of mo-DC. Four preparations of DC from CD34+ precursors are able to support high-level, stable expression of genes driven by the HIV-1 LTR, indicating that sufficient Sp1 or compensatory transcriptional factors are present in these cells. The transduced genes are likely to be integrated since they are also expressed in other subsets of progeny cells, such as macrophages and erythrocytes.

### EXAMPLE 5

### Fully Functional Immature DC Cultured From Blood Monocytes (Mo-DC) Will Generate HIV-Specific CD3+CTL

To generate fully functional immature DC, adherent monocytes are in GM-CSF and IL-4 for 5 d before infection with the HIV-1 vector uniquely capable of integrating into noncycling cells. Previous experiments have demonstrated that mo-DC efficiently expressed the GFP reporter gene driven from the HIV LTR (see above). The mo-DC are routinely >95% homogeneous and up to 10⁹ cells can be prepared from each leukapheresis sample. CD8+ T cells are positively selected using immunomagnetic beads. T cells are isolated with a CD8 peptide₅₉₋₇₀-specific monoclonal antibody and eluted from the magnetic beads with the corresponding peptide. Nonspecific activation with this procedure has been noted and the purified T cells were more homogeneous than preparations isolated by negative selection. CD8+ T cells are admixed with virus-transduced DC (v-DC) at a ratio of 10:1 and incubated for 4 d. Selective expansion of virus-specific T cells is performed in a low dose of IL-2 and IL-7 with weekly restimulation with v-DC plus cytokines for up to 7 weeks. Virus-specific cytotoxicity is determined by a standard chromium release assay, using virus-infected HLA A2.1-expressing Jurkat cells (A2.1-Jurkat) as positive targets and for negative control targets, the uninfected A2.1 Jurkat as well as A2.1 melanoma cell. To determine whether the CTL response is broadly specific, the ability of the T cells to lyse A2.1-Jurkat pulsed with known A2.1-restricted epitopes of gag and pol is tested (e.g., pol: p476-484; p652-660; gag, p77-85). In the event that the virus-specific CTL reactivity was not directed to known epitopes, the novel epitopes are identified by pulsing A2.1-Jurkat cells with a panel of nanomeric peptides overlapping by three residues encoding known immunogenic regions of gag, pol or accessory proteins (e.g., rev, tat, vif). These stably transduced mo-DC can be used for repeated immunization in vivo or for ex vivo priming of CTL for adoptive T cell therapy.

### EXAMPLE 6

### Populations of Fresh (Uncultured) DC and Other Committed DC Precursors are Effective Antigen Presenting Cells After Virus Transuction

Peripheral blood mononuclear cells (PBMC) are obtained from volunteers given G-CSF. Fresh DC populations are isolated by density gradient centrifugation followed by immunodepletion of nonmyeloid lineage cells. Both fresh mature and immature DC, distinguished by their ability to present alloantigens in a mixed lymphocyte reaction (MLR) and expression of accessory molecules, are transduced with the lentiviral vectors. The ability of untransduced immature DC to differentiate into immunocompetent DC by MLR and expression of appropriate costimulatory and accessory molecules is then determined. If necessary, cytokines will be incorporated into the viral vectors such as flt3 ligand (FL) or IL-4 that has been shown to induce maturation of these DC precursors. Ultimately, all transduced DC are tested for their ability to generate HIV-specific CTL.

### EXAMPLE 7

### In Vivo Transduction in a Mouse Model

Mice are immunized with syngeneic DC transduced with the HIV vector containing the CMV promoter and virus-specific CTL activity is measured in the spleen and lymph mode. For proof of concept of in vivo transduction, mice are immunized in vivo with the vector, after daily injections of FL designed to increase the umber of DC precursors in vivo. Treatment of mice with Flt3 ligand (Flt3L) greatly increased the numbers of different subpopulations of functionally mature dendritic cells (Maraskovsky et al., J. Exp. Med. 184:1953, 1997). Immune responses in mice generated by in vivo transduction of with or without Flt3L treatment with HIV vectors expressing Env antigens are compared.

Eight week old female Balb/c mice are injected subcutaneously once daily with either mouse serum albumin (MSA) (1 μg) or with MSA plus 10 μg of ft3L for nine consecutive days. At days 0 and 7, mice are injected with VSV(G)/HIV-1 vectors (10e8 TCID₅₀/animal). On day 17, blood is collected to test for env binding and virus neutralizing antibodies, and splenocytes are isolated to test for CTL activity or CD4 helper activity. DCs are also expanded from the bone marrow and tested for APC function. For CTL assays, BALB/c.3T3 fibroblasts are transduced with the VSV/HIV-1 vector to be used as target cells. For CD4 T-helper activity, the splenocytes are restimulated in vitro with autologous, irradiated, vector transduced DC for three days, and assayed for proliferation by ³H-thymidine incorporation and cytokine production using cytokine ELISAs (IL2 or γ-IFN for T_{H}1 response, IL4 for T_{H}2 response). ELISA antibodies to MN gp120 and neutralizing antibodies against laboratory strains MN, IIIB, SF2) and primary isolates are measured. To determine whether the T helper response is Type I or II, cytokine production by splenocytes is determined by intracellular staining with cytokine-specific mAb after treating for 4 hours with PMA (20 ng/ml) plus ionomycin (1 μm) in the presence of monensin. Cells are then fixed, permeabilized, and stained with Cy-chrome anti-mouse CD4, FITC anti-mouse gamma IFN and PE anti-mouse IL-4 for analysis by flow cytometry.

To mobilize DC with FL, mice are injected subcutaneously once daily with 10 ug of FL for nine consecutive days. On days 0 and 7, mice are injected with VSV(G)/HIV-1 vectors (10e8 TCID₅₀/animal). On day 17, blood will be collected to test for Env binding and virus neutralizing antibodies, and splenocytes are isolated to test for CTL and CD4 T helper cell activity.

It will be apparent to those skilled in the art that various modifications and variations can be made to the compositions and processes of this invention. Thus, it is intended that the present invention cover such modifications and variations, provided they come within the scope of the appended claims and their equivalents.

What is claimed is

1. A method of inducing an immune response in a subject, comprising:
administering to the subject, a therapeutically effective amount of a dendritic cell or a progenitor thereof, transduced with a replication defective pseudotyped lentiviral vector comprising a nucleic acid sequence encoding an antigen such that the antigen is presented on the surface of the dendritic cell.

2. The method of claim 1, wherein the dendritic cell is an immature dendritic cell.

3. The method of claim 1, wherein the dendritic cell is a non-dividing dendritic cell.

4. The method of claim 1, wherein the progenitor of a dendritic cell is a CD34⁺ cell.

5. The method of claim 1, wherein the pseudotyped lentiviral vector comprises a nucleic acid encoding a cytokine.

6. The method of claim 5, wherein the cytokine is selected from the group consisting of interleukin-2 (IL-2), interleukin-3 (IL-3), interleukin-4 (IL-4), Flt-3/Flk-2 ligand (FL), granulocyte macrophage colony stimulating factor (GM-CSF) and stem cell factor (SCF).

7. The method of claim 1, wherein the antigen is a tumor antigen.

8. The method of claim 1, wherein the antigen is a fusion polypeptide comprising more than one antigen.

9. The method of claim 1, wherein the antigen is a lentiviral antigen or a fragment thereof.

10. The method of claim 9, wherein the lentiviral antigen is a Human Immunodeficiency Virus (HIV-1) antigen.

11. The method of claim 9, wherein the lentiviral antigen is selected from the group consisting of the gag, pol, env, vpr, vif nef, vpx, tat, rev, vpu gene products and fragments thereof.

12. The method of claim 1, wherein the pseudotyped lentiviral vector contains an envelope protein selected from the group consisting of a vesicular stomatitis virus G (VSV-G) protein and a moloney leukemia virus (MLV) protein.

13. The method of claim 1, wherein the pseudotyped lentiviral vector is a human immunodeficiency virus (HIV-1) vector.

14. The method of claim 1, wherein the pseudotyped lentiviral vector is a non-HIV lentiviral vector.

15. A method of inducing an immune response in a subject, comprising:
transducing a dendritic cell or a progenitor of a dendritic cell with a pseudotyped lentiviral vector comprising a nucleic acid sequence encoding an antigen such that the antigen is presented on the surface of the dendritic cell to produce a transduced dendritic cell; and
contacting the transduced dendritic cells with a T cell to produce an activated T cell, wherein at least one of the pseudotyped lentiviral vector, the transduced dendritic cell and the T cell, are administered to the subject.

16. The method of claim 15, wherein the transducing occurs in vivo.

17. The method of claim 15, wherein the transducing occurs in vitro.

18. The method of claim 15, wherein the contacting occurs in vivo.

19. The method of claim 15, wherein the contacting occurs in vitro.

20. The method of claim 15, wherein the dendritic cell is an immature dendritic cell.

21. The method of claim 15, wherein the dendritic cell is non-dividing dendritic cell.

22. The method of claim 15, wherein the progenitor of a dendritic cell is a CD34⁺ cell.

23. The method of claim 15, wherein the pseudotyped lentiviral vector comprises a nucleic acid encoding a cytokine.

24. The method of claim 23, wherein the cytokine is a member selected from group consisting of interleukin-2 (IL-2), interleukin-3 (IL-3), interleukin-4 (IL-4), Flt-3/Flk-2 ligand (FL), granulocyte macrophage colony stimulating factor (GM-CSF) and stem cell factor (SCF).

25. The method of claim 15, wherein the antigen is a tumor antigen.

26. The method of claim 15, wherein the antigen is a fusion polypeptide comprising more than one antigen.

27. The method of claim 15, wherein the antigen is a lentiviral antigen or a fragment thereof.

28. The method of claim 27, wherein the lentiviral antigen is a Human Immunodeficiency Virus (HIV-1) antigen.

29. The method of claim 27, wherein the lentiviral antigen is selected from the group consisting of the gag, pol, env, vpr, vif nef, vpx, tat, rev, vpu gene products and fragments thereof.

30. The method of claim 15, wherein the pseudotyped lentiviral vector contains an envelope protein selected from the group consisting of a vesicular stomatitis virus G (VSV-G) protein and a moloney leukemia virus (MLV) protein.

31. The method of claim 15, wherein the pseudotyped lentiviral vector is a human immunodeficiency virus (HIV-1) vector.

32. The method of claim 15, wherein the pseudotyped lentiviral vector is a non-HIV lentiviral vector.

33. A method of activating a T cell comprising contacting a T cell with a dendritic cell having an antigen on its surface, wherein the dendritic cell comprises a pseudotyped lentiviral vector comprising a nucleic acid sequence encoding the antigen, wherein the contacting results in activating the T cell.

34. The method of claim 33, wherein the dendritic cell is an immature dendritic cell.

35. The method of claim 33, wherein the dendritic cell is a non-dividing dendritic cell.

36. The method of claim 33, wherein the progenitor of a dendritic cell is a CD34⁺cell.

37. The method of claim 33, wherein the activating occurs in vivo.

38. The method of claim 33, wherein the activating occurs in vitro.

39. The method of claim 33, wherein the pseudotyped lentiviral vector comprises a nucleic acid encoding a cytokine.

40. The method of claim 39, wherein the cytokine is selected from the group consisting of interleukin-2 (IL-2), interleukin-3 (IL-3), interleukin-4 (IL-4), Flt-3/Flk-2 ligand (FL), granulocyte macrophage colony stimulating factor (GM-CSF) and stem cell factor (SCF).

41. The method of claim 33, wherein the antigen is a tumor antigen.

42. The method of claim 33, wherein the antigen is a ftision polypeptide comprising more than one antigen.

43. The method of claim 33, wherein the antigen is a lentiviral antigen or a fragment thereof.

44. The method of claim 43. wherein the lentiviral antigen is a Human Immunodeficiency Virus (HIV-1) antigen.

45. The method of claim 43, wherein the lentiviral antigen is selected from the group consisting of the gag, pol, env, vpr, vif, nef, vpx, tat, rev, vpu gene products and fragments thereof.

46. The method of claim 33, wherein the pseudotyped lentiviral vector contains an envelope protein selected from the group consisting of a vesicular stomatitis virus G (VSV-G) protein and a moloney leukemia virus (MLV) protein.

47. The method of claim 33, wherein the pseudotyped lentiviral vector is a human immunodeficiency virus (HIV-1) vector.

48. The method of claim 33, wherein the pseudotyped lentiviral vector is a non-HIV lentiviral vector.
